(19) United States
(12) Patent Application Publication
(10) Pub. No.: US 2022/0226035 A1
(43) Pub. Date: Jul. 21, 2022

(54) Electrosurgical Generator and Method of Generating Electrosurgical Energy

(71) Applicant: Stryker European Operations Limited, Cork (IE)

(72) Inventors: Steen MØLLER, Frederikssund (DK); Joakim SOYA, Stenløse (DK)

(21) Appl. No.: 17/615,496

(22) PCT Filed: May 29, 2020
(86) PCT No.: PCT/IB2020/000433

§ 371 Date: Nov. 30, 2021

Related U.S. Application Data
(60) Provisional application No. 62/854,380, filed on May 30, 2019.

(51) Int. Cl. A61B 18/12

(52) U.S. Cl. CPC A61B 18/1206; A61B 2018/00892

(57) ABSTRACT

In an example, an electrosurgical generator includes a power converter configured to convert a supply power received from a power source to an output power. The output power is suitable for delivering electrosurgical energy. The electrosurgical generator also includes a current sensor configured to sense a current of the output power and generate a logarithmic and analog representation of the current, and a voltage sensor configured to sense a voltage of the output power and generate a logarithmic and analog representation of the voltage. The electrosurgical generator further includes a controller configured to: (i) receive the logarithmic and analog representation of the current sensed by the current sensor, (ii) receive the logarithmic and analog representation of the voltage sensed by the voltage sensor, and (iii) adjust, based on the logarithmic and analog representation of the current and the voltage, a voltage

### CROSS-REFERENCE TO RELATED APPLICATIONS

The present application claims the benefit of priority to U.S. Provisional Application No. 62/854,380, filed May 30, 2019, the contents of which is hereby incorporated by reference in its entirety.

### FIELD

The present disclosure generally relates to electrosurgery and, in particular, to an electrosurgical generators and methods of generating electrosurgical energy.

### BACKGROUND

Electrosurgery involves applying a radio frequency (RF) electric current (also referred to as electrosurgical energy) to biological tissue to cut, coagulate, or modify the biological tissue during an electrosurgical procedure. Specifically, an electrosurgical generator generates and provides the electric current to a first electrode, which applies the electric current (and, thus, electrical power) to the tissue. The electric current passes through the tissue and returns to the generator via a second electrode. As the electric current passes through the tissue, an impedance of the tissue converts a portion of the electric current into thermal energy (e.g., via the principles of resistive heating), which increases a temperature of the tissue and induces modifications to the tissue (e.g., cutting, coagulating, ablating, and/or sealing the tissue). Accordingly, an extent to which the tissue is affected by the electrosurgery is related to the electrical power transmitted to the tissue.

### BRIEF DESCRIPTION OF THE FIGURES

The novel features believed characteristic of the illustrative examples are set forth in the appended claims. The illustrative examples, however, as well as a preferred mode of use, further objectives and descriptions thereof, will best be understood by reference to the following detailed description of an illustrative example of the present disclosure when read in conjunction with the accompanying drawings, wherein:

FIG. 1 depicts an electrosurgical system, according to an example.

FIG. 2 depicts a simplified block diagram of an electrosurgical system, according to an example.

FIG. 3 is a plot of a plotline representing a target power over a range of impedance values, according to an example.

FIG. 4A depicts a current measured for a representative feedback cycle, according to an example.

FIG. 4B depicts a voltage measured for the feedback cycle of FIG. 4A, according to an example.

FIG. 5 depicts a simplified block diagram of an electrosurgical system, according to another example.

FIG. 6 is a flowchart of a method of generating electrosurgical energy, according to an example.

FIG. 7 is a flowchart of a method of generating electrosurgical energy for use with the method of FIG. 6, according to an example.

FIG. 8 is a flowchart of a method of generating electrosurgical energy for use with the method of FIG. 6, according to an example.

FIG. 9 is a flowchart of a method of generating electrosurgical energy for use with the method of FIG. 8, according to an example.

FIG. 10 is a flowchart of a method of generating electrosurgical energy for use with the method of FIG. 8, according to an example.

FIG. 11 is a flowchart of a method of generating electrosurgical energy for use with the method of FIG. 8, according to an example.

FIG. 12 is a flowchart of a method of generating electrosurgical energy for use with the method of FIG. 6, according to an example.

FIG. 13 is a flowchart of a method of generating electrosurgical energy for use with the method of FIG. 6, according to an example.

FIG. 14 is a flowchart of a method of generating electrosurgical energy for use with the method of FIG. 13, according to an example.

FIG. 15 is a flowchart of a method of generating electrosurgical energy for use with the method of FIG. 13, according to an example.

FIG. 16A is a first portion of a flowchart of a method of generating electrosurgical energy, according to an example.

FIG. 16B is a second portion of the flowchart of a method shown in FIG. 16B, according to an example.

### DETAILED DESCRIPTION

Disclosed examples will now be described more fully hereinafter with reference to the accompanying drawings, in which some, but not all of the disclosed examples are shown. Indeed, several different examples may be described and should not be construed as limited to the examples set forth herein. Rather, these examples are described so that this disclosure will be thorough and complete and will fully convey the scope of the disclosure to those skilled in the art.

By the term “approximately” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

As noted above, an extent to which the tissue is affected by the electrosurgery is related to the electrical power transmitted to the tissue. An electrosurgical generator may include sensors that can detect a voltage and a current of the electrosurgical energy transmitted to the tissue and use the voltage and the current as a basis for estimating the electrical power transmitted to the tissue. The electrosurgical generator can then use the estimated power as a basis for controlling a power of the electrosurgical energy generated by the electrosurgical generator during an electrosurgical procedure.

Conventionally, the electrosurgical generator digitally samples the current and the voltage at a relatively high frequency. This can result in a loss of information that was contained in original analog signals of the current and voltage that were digitally sampled, which can impair the accuracy of the estimated power and the control of the power by the electrosurgical generator.

Within examples, the present disclosure provides for an electrosurgical generator that can address at least some of the drawbacks described above. For instance, within examples, the present disclosure provides for an electrosurgical generator that can use analog measurements of the current and the voltage as a basis for determining the power of the electrosurgical energy transmitted to the tissue. By using analog measurements, the electrosurgical generator does not suffer from the loss of information associated with the conventional approaches described above. Accordingly, the electrosurgical generator can more precisely and accurately control the power of the electrosurgical energy to better achieve a desired clinical effect.

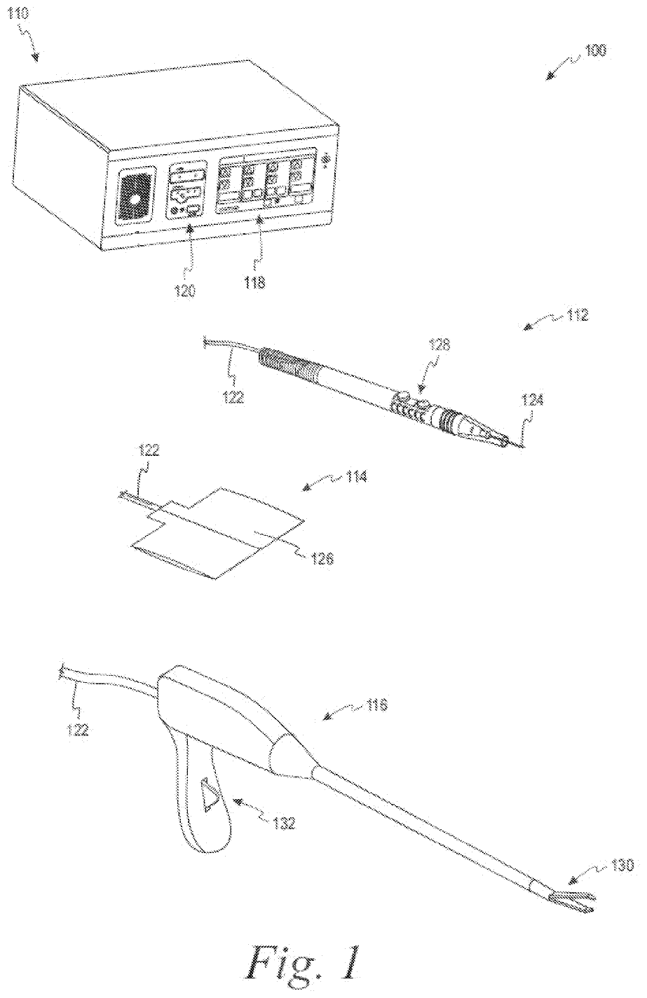

Referring now to FIG. 1, an electrosurgical system 100 is shown according to an example. As shown in FIG. 1, the electrosurgical system 100 can include an electrosurgical generator 110 and one or more electrosurgical tools 112, 114, 116. In general, the electrosurgical generator 110 can generate electrosurgical energy that is suitable for performing electrosurgery on a patient. More particularly, as described in detail below, the electrosurgical generator 110 can include a power converter that can convert a supply power (e.g., grid power) to an output power, which is suitable for delivering electrosurgical energy to the patient. Also, as described in detail below, the electrosurgical generator 110 can include one or more electrical components that can control a voltage, a current, and/or a frequency of the output power to achieve a desired clinical effect.

In one example, the output power can have a frequency that is greater than approximately 100 kilohertz (KHz) to reduce (or avoid) stimulating a muscle and/or a nerve near the target tissue. In another example, the output power can have a frequency that is between approximately 300 kHz and approximately 500 kHz. In another example, the output power can have a frequency between approximately 440 kHz and approximately 500 kHz. In another example, the output power can have a frequency of approximately 472 kHz.

The electrosurgical generator 110 can be operable in a plurality of modes of operation. As examples, the modes of operation can include a cutting mode, a coagulating mode, an ablating mode, and/or a sealing mode. The modes of operation can correspond to respective levels of power and/or respective waveforms for the output power. Thus, within examples, the electrosurgical generator 110 can generate the output power with a level of power and/or a waveform respectively selected from a plurality of levels of power and/or a plurality of waveforms based on the mode of operation in which the electrosurgical generator 110 is operated.

Within examples, the electrosurgical generator 110 can include a user interface 118 that can receive one or more inputs from a user and/or provide one or more outputs to the user. As examples, the user interface 118 can include one or more buttons, one or more switches, one or more dials, one or more keypads, one or more touchscreens, and/or one or more display screens. In an example, the user interface 118 can be operable to select a mode of operation from among the plurality of modes of operation and/or set a level of power for one or more modes of operation for the electrosurgical generator 110. As such, the electrosurgical generator 110 can generate the output power with the level of power and/or the waveform respectively selected from the plurality of levels of power and/or the plurality of waveforms based, at least in part, on one or more inputs received via the user interface 118.

As shown in FIG. 1, the electrosurgical generator 110 can also include at least one tool input 120 that can facilitate coupling the electrosurgical generator 110 to the one or more electrosurgical tools 112, 114, 116. In an example, each electrosurgical tool 112, 114, 116 can include an electrical conductor 122 having a plug, which can be coupled to a respective socket of the at least one tool input 120. In this arrangement, the electrosurgical generator 110 can supply the output power to the one or more electrosurgical tools 112, 114, 116 via the coupling between the at least one tool input 120 of the electrosurgical generator 110 and the electrical conductor 122 of the one or more electrosurgical tools 112, 114, 116.

In FIG. 1, the one or more electrosurgical tools 112, 114, 116 include a monopolar electrosurgical tool 112, a dispersive electrode pad 114, and/or a bipolar electrosurgical tool 116. The monopolar electrosurgical tool 112 and the dispersive electrode pad 114 can be used to operate the electrosurgical system 100 in a monopolar mode. Whereas, the bipolar electrosurgical tool 116 can be used to operate the electrosurgical system 100 in a bipolar mode.

For example, to operate the electrosurgical system 100 in the monopolar mode, the monopolar electrosurgical tool 112 and the dispersive electrode pad 114 can be coupled to respective sockets of the at least one tool input 120 of the electrosurgical generator 110. The electrosurgical generator 110 can supply electrosurgical energy by providing the output power to the monopolar electrosurgical tool 112. The monopolar electrosurgical tool 112 can include an active electrode 124 for applying the electrosurgical energy to a target tissue of a patient, and the dispersive electrode pad 114 can include a neutral electrode 126 (also referred to as a “dispersive electrode”) for returning the electrosurgical energy from the target tissue to the electrosurgical generator 110. The dispersive electrode pad 114 can contact the patient with a surface area that is suitable to mitigate a risk of unintended tissue damage due to the electrosurgical energy flowing through the tissue (i.e., damage to the tissue other than the target tissue).

As shown in FIG. 1, in some examples, the monopolar electrosurgical tool 112 can include at least one user input device 128 that can select between the modes of operation of the electrosurgical generator 110. For instance, in one implementation, the at least one user input device 128 can be configured to select between a cutting mode of operation and a coagulation mode of operation. Responsive to actuation of the at least one user input device 128 of the monopolar electrosurgical tool 112, the monopolar electrosurgical tool 112 can (i) receive the output power with a level of power and/or a waveform corresponding to the mode of operation selected via the at least one user input device 128 and (ii) supply the output power to the active electrode 124.

To operate the electrosurgical system 100 in the bipolar mode, the bipolar electrosurgical tool 116 can be coupled to a respective socket of the at least one tool input 120 of the electrosurgical generator 110. The bipolar electrosurgical tool 116 can have two active electrodes 130, and the target tissue can be positioned between the active electrodes. When the electrosurgical generator 110 supplies the electrosurgical energy, (i) one of the active electrodes 130 applies the electrosurgical energy to the target tissue and (ii) the other one of the active electrodes 130 returns the electrosurgical energy to the electrosurgical generator 110.

In some examples, the bipolar electrosurgical tool 116 can include at least one user input device 132 for controlling supply of the electrosurgical energy from the electrosurgical generator 110 to the active electrodes 130 (e.g., starting and/or stopping supplying the electrosurgical energy to the active electrodes 130). In other examples, the electrosurgical system 100 can additionally or alternatively include a footswitch for controlling supply of the electrosurgical energy from the electrosurgical generator 110 to the active electrodes 130.

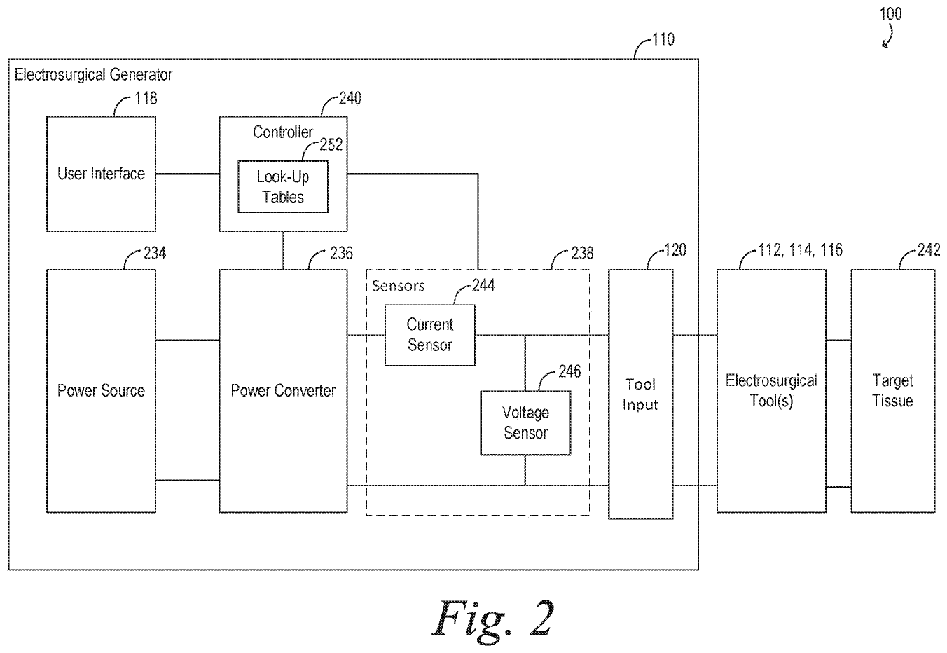

Referring now to FIG. 2, a simplified block diagram of the electrosurgical system 100 is shown according to an example. As shown in FIG. 2, the electrosurgical system 100 includes the electrosurgical generator 110 and the one or more electrosurgical tools 112, 114, 116. Also, as described above, the electrosurgical generator 110 can include the user interface 118 and the at least one tool input 120.

As shown in FIG. 2, the electrosurgical generator 110 can also include a power source 234, a power converter 236, a plurality of sensors 238, and a controller 240. The power source 234 is coupled to the power converter 236, and the power converter 236 is coupled to the one more electrosurgical tools 112, 114, 116 via the at least one tool input 120. Within examples, the power source 234 can be a grid power, a backup generator, a power storage device (e.g., a battery), and/or a renewable power source (e.g., a solar power source, a hydroelectric power source, and/or a windmill). Although FIG. 2 shows the electrosurgical generator 110 including the power source 234, the power source 234 can be separate from the electrosurgical generator 110 in other examples.

The power converter 236 can convert a supply power received from the power source 234 to the output power, which is suitable for delivering electrosurgical energy to a target tissue 242. As shown in FIG. 2, the power converter 236 is coupled to the controller 240. The controller 240 can communicate with the power converter 236 (e.g., by transmitting one or more control signals to the power converter 236) to control one or more electrical parameters of the output power such as, for example, a voltage, a current, a frequency, a waveform, and/or a duty cycle of the output power. The controller 240 can additionally or alternatively cause the power converter 236 to start and/or stop supplying the output power to the one or more electrosurgical tools 112, 114, 116.

Within examples, the controller 240 can set and/or adjust the electrical parameter(s) of the output power based on the mode of operation and/or the one or more inputs received via the user interface 118. Additionally, the controller 240 can set and/or adjust the electrical parameter(s) of the output power based on feedback information received from the sensors 238. Accordingly, as shown in FIG. 2, the controller 240 can be in communication with the sensors 238.

As shown in FIG. 2, the sensors 238 can include a current sensor 244 and a voltage sensor 246. The current sensor 244 can sense a current of the output power and generate a logarithmic and analog representation of the current. For instance, in FIG. 2, the current sensor 244 can be a logarithmic root-mean-square (RMS) current detector that is coupled in series with a first conductor 248 transmitting the output power from the power converter 236 to the one or more electrosurgical tools 112, 114, 116 via the at least one tool input 120. Thus, in this example, the current sensor 244 can sense the current of the output power as a RMS current value. Sensing the current of the output power as the RMS current value can help to simplify one or more calculations performed by the controller 240.

The voltage sensor 246 can sense a voltage of the output power and generate a logarithmic and analog representation of the voltage. For instance, in FIG. 2, the voltage sensor 246 can be a logarithmic RMS voltage detector that is coupled in parallel with the power converter 236 (e.g., coupled between the first conductor 248 and a second conductor 250, which transmits the electrosurgical energy from the one or more electrosurgical tools 114, 116 back to the electrosurgical generator 110 via the at least one tool input 120). Thus, in this example, the current sensor 244 can sense the current of the output power as a RMS voltage value. Sensing the voltage of the output power as the RMS voltage value can help to simplify one or more calculations performed by the controller 240.

As noted above, the controller 240 can set and/or adjust the electrical parameter(s) of the output power based on feedback information received from the current sensor 244 and the voltage sensor. More particularly, the controller 240 is configured to (i) receive the logarithmic and analog representation of the current sensed by the current sensor 244, (ii) receive the logarithmic and analog representation of the voltage sensed by the voltage sensor 246, and (iii) adjust, based on the logarithmic and analog representation of the current and the voltage, a voltage of the output power.

In one example, the controller 240 can determine whether to adjust the voltage and/or determine an amount to adjust the voltage of the output power based on a plurality of the logarithmic and analog representations of the current and a plurality of the logarithmic and analog representations of the voltage over a sampling interval. This can allow the controller 240 to adjust the voltage of the output power based on a greater amount of information, which can help the electrosurgical generator 110 deliver the output power to the target tissue 242 with greater accuracy and precision and, thus, better achieve the desired clinical effect.

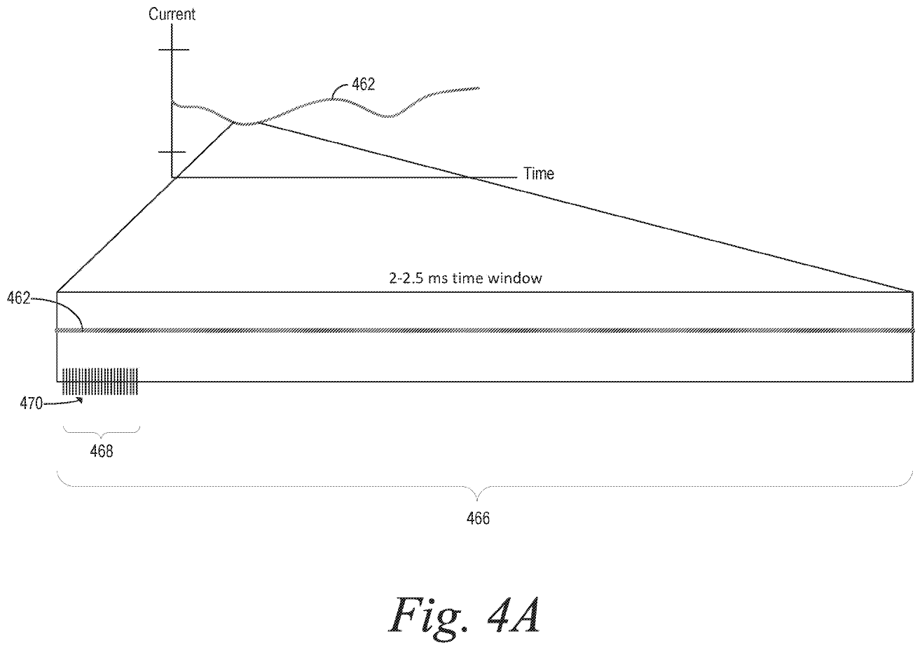

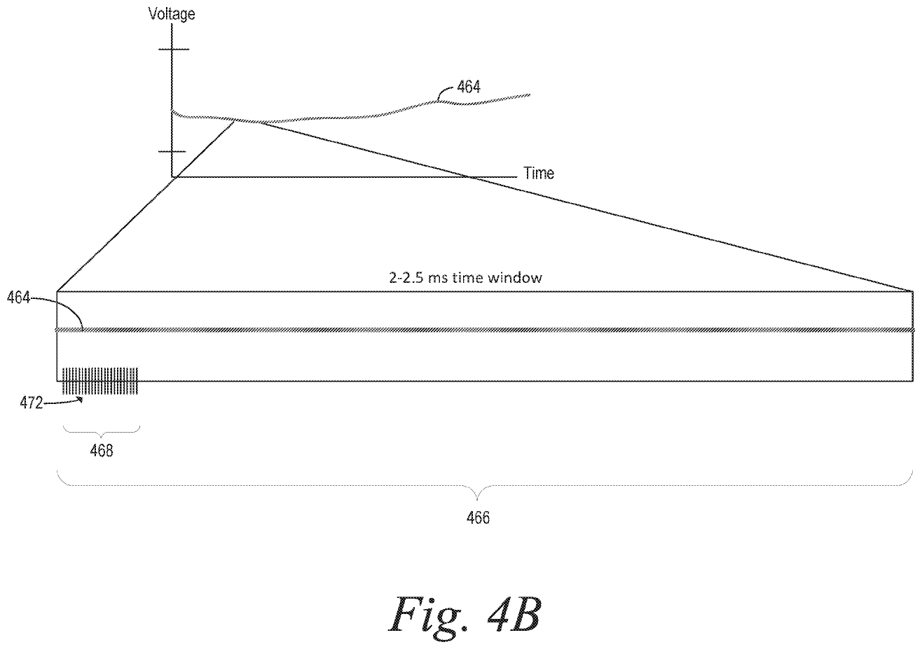

In an implementation, to adjust the voltage of the output power, the controller can determine, based on the logarithmic and analog representation of the current, a plurality of analog current values at a plurality of time points during a sampling interval, and determine, based on the logarithmic and analog representation of the voltage, a plurality of analog voltage values at the plurality of time points during the sampling interval. Additional details relating to the controller 240 determining the plurality of analog current values, the plurality of analog voltage values, the average current, and the average voltage will be described below with respect to FIGS. 4A-4B.

The controller 240 can then determine an average current value by averaging the plurality of analog current values, and determine an average voltage value by averaging the plurality of analog voltage values. For example, to determine the average current value, the controller 240 can sum the plurality of analog current values and divide the sum by a quantity of the plurality of analog current values. Similarly, to determine the average voltage value, the controller 240 can sum the plurality of analog voltage values and divide the sum by a quantity of the plurality of analog voltage values.

In an example, to facilitate summing the analog current values and the analog voltage values, the controller 240 can store digital representations of the analog current values and the analog voltage values in a memory of the controller 240. In such example, the controller 240 can include one or more analog-to-digital converters (ADC) to convert the analog current values and the analog voltage values to the digital representations. In contrast to conventional approaches that digitally sample the current and the voltage, the electrosurgical generator 110 can determine analog measurements of the current and the voltage at discrete time points and then store the analog measurements as digital values. As such, unlike the conventional approaches, the electrosurgical generator 110 does not suffer from the same loss of information challenges encountered using such conventional approaches.

After determining the average current value and the average voltage value, the controller 240 can use the average current value and the average voltage value as a basis for determining whether to adjust the voltage of the output power and/or the amount to adjust the voltage of the output power. For instance, the controller 240 can determine, based on the average current value and the average voltage value, at least one of a power value or an impedance value. The controller 240 can determine the power value using equation 1 below:

$$P = {V}_{avg} * {I}_{avg} \qquad ( eq . 1 )$$

where P is the power value, V_{avg} is the average voltage value, and I_{avg} is the average current value. The controller 240 can determine the impedance value using Ohm's law represented by equation 2 below:

$$Z = {V}_{a v g} / {I}_{a v g} \qquad ( eq . 2 )$$

where Z is the impedance value, V_{avg} is the average voltage value, and I_{avg} is the average current value. After determining the power value and the impedance value, the controller 240 can adjust, based on the at least one of the power value or the impedance value, the voltage of the output power.

For example, the controller 240 can store a plurality of target power values and a plurality of measured impedance values, where each target power value corresponds to a respective one of the measured impedance values. The target power value can relate to a desired power of the output power for a measured impedance of the target tissue, which can achieve a desired clinical outcome during electrosurgery. In some examples, the target power values can be based on one or more user inputs received via the user interface 118 of the electrosurgical generator 110.

In practice, the power value determined by the controller 240 can deviate from the target power value corresponding to the impedance value determined by the controller 240. The controller 240 can be configured to adjust the voltage of the output power to compensate for the difference between the power value (i.e., the actual measured power of the output power) and the target power value for the impedance value (i.e., the actual measured impedance of the target tissue).

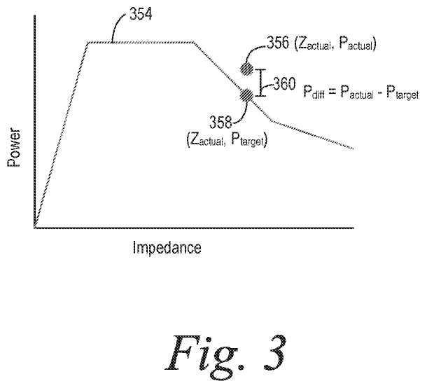

To illustrate, FIG. 3 depicts a graphical representation of the target power values plotted against a plurality of impedance values, according to an example. In particular, FIG. 3 shows a plotline 354 that indicates the target power value that corresponds to each impedance value for this example. In FIG. 3, a measured data point 356 shows the actual power value (P_{actual}) and the impedance value (Z_{actual}) determined by the controller 240 for an example feedback cycle of the controller 240 (e.g., based on the average current and the average voltage as described above).

As shown in FIG. 3, the measured data point 356 is not on the plotline 354. Rather, in FIG. 3, the plotline 354 includes a target data point 358 corresponding to a target power (P_{target}) at the impedance value (Z_{actual}). Accordingly, as shown in FIG. 3, there is a power difference 360 (P_{diff}) between (i) the actual power (P_{actual}) determined by the controller 240 at the impedance value determined by the controller 240 (Z_{actual}), and (ii) the target power (P_{target}) at the impedance value determined by the controller 240 (Z_{actual}).

Within examples, the controller 240 can adjust the power of the output power to reduce or eliminate the power difference (P_{diff}) between the actual power (P_{actual}) and the target power (P_{target}). In one implementation, the controller 240 can first decide based on the impedance value and the power value whether to adjust the power of the output power or maintain the power of the output power. For instance, the controller 240 can look up the impedance value in a table 252 to identify a target power value that corresponds to the impedance value, and perform a comparison of the power value determined by the controller 240 and the target power value to determine whether to adjust or maintain the power of the output power. In an example, the controller 240 can compare the power value and the target power value by determining a difference between the power value and the target value. If the controller 240 determines that the difference is less than the threshold value, the controller 240 can decide to maintain the voltage of the output power. Whereas, if the controller 240 determines that the difference is greater than a threshold value, the controller 240 can decide to adjust the voltage of the output power. As an example, the threshold value can be between 0 percent and approximately 5 percent of the target power value.

If the decision is to adjust the power of the output power, then the controller 240 can also determine, based on the comparison, an adjusted voltage value. As one example, the controller 240 can determine the adjusted voltage value based on the target power value and the power value determined by the controller 240 (i.e., based on the average current and the average voltage). The controller 240 can then adjust the voltage of the output power to the adjusted voltage value. Responsive to the controller 240 deciding to adjust the voltage of the output power and/or determining the adjusted voltage value, the controller 240 can transmit a control signal to the power converter 236 to cause the power converter 236 to adjust the voltage of the output power (e.g., adjust the voltage of the output power to the adjusted voltage value). For instance, the controller 240 can (i) reduce the voltage of the output power responsive to the controller 240 determining that the power value is greater than the target power value, and (ii) increase the voltage of the output power responsive to the controller 240 determining that the power value is less than the target power value.

In another implementation, to adjust the voltage of the output power, the controller 240 can additionally or alternatively be configured to look up the power value and the impedance value (determined by the controller 240 as described above) in the table 252 to identify an adjusted voltage value that corresponds to the power value and the impedance value, and the controller 240 can adjust the voltage of the output power to the adjusted voltage value. The table 252 can be stored in the memory of the controller 240. The table 252 can map various adjusted voltage values to corresponding power values and impedance values. For instance, each adjusted voltage value can relate to a level of voltage that can achieve the target power corresponding to a respective one of the impedance values. In this arrangement, the controller 240 can be programmed to refer to the table 252 to select the adjusted voltage value that corresponds to the combination of the power value and the impedance value determined by the controller 240 based on the average current value and the average voltage value.

Within examples, the controller 240 can (i) determine the analog current values, the analog voltage values, the average current value, the average voltage value, the impedance value, and/or the power value and (ii) adjust or maintain the power of the output power on a periodic basis during an electrosurgery procedure. For instance, the controller 240 can perform the above-described operations during each feedback cycle of a series of feedback cycles to control the power of the output power during the electrosurgery procedure. In one implementation, each feedback cycle be performed within a respective time window having a duration between approximately 2 milliseconds (ms) and approximately 2.5 ms. In this implementation, the sampling interval during which the controller 240 determines the analog current values and the voltage current values can occur for only a portion of the time window such as, for example, over a period of time of approximately 132 microseconds.

FIGS. 4A-4B graphically depict a representative feedback cycle that can be performed by the controller 240, according to an example. Specifically, FIG. 4A depicts a plot of the logarithmic and analog representations of the current 462 sensed by the current sensor 244 over time, and FIG. 4B depicts a plot of the logarithmic and analog representations of the voltage 464 sensed by the voltage sensor 246 over time. Additionally, FIGS. 4A-4B show a time window 466 having a duration of approximately 2 ms to approximately 2.5 ms.

During a sampling interval 468 of the time window 466, the controller 240 can determine the analog current values 470 and the analog voltage values 472. In this example, the controller 240 can determine 32 analog current values 470 and 32 analog voltage values 472, and the sampling interval is approximately 132 microseconds. However, the controller 240 can determine a lesser quantity or a greater quantity of the analog current values 470 and the analog voltage values 472 during the sampling interval 468 in other examples. Similarly, in other examples, the sampling interval 468 can be a lesser amount of time or a greater amount of time than 132 microseconds.

As noted above, the analog current values 470 and the analog voltage values 472 can be RMS current values and RMS voltage values in one example. Also, as described above, the controller 240 can then average the analog current values by summing the analog current values and dividing the sum by the quantity of samples (i.e., 32). Similarly, the controller 240 can average the analog voltage values by summing the analog voltage values and dividing the sum by the quantity of samples (i.e., 32). The controller 240 can then use the average current value and the average voltage value to determine the impedance value and/or the power value, and use the impedance value and/or the power value as a basis for adjusting or maintaining the power of the output power for the feedback cycle.

Within examples, the controller 240 can cause the power converter 236 to adjust the voltage of the output power responsive to the controller 240 making a decision to adjust the power of the output power. As shown in FIGS. 4A-4B, after determining the analog current values 470 and the analog voltage values 472, the controller 240 can wait for a period of time for the feedback cycle to end, and a next feedback cycle in the series to begin. When the next feedback cycle begins, the controller 240 can repeat the process of (i) determining the analog current values 470, the analog voltage values 472, the average current value, the average voltage value, the impedance value, and/or the power value, and (ii) adjusting or maintaining the power of the output power. As described above, the controller 240 can perform the series of feedback cycles on a periodic basis and, thus, the next feedback cycle can occur over another time window of approximately 2 ms to approximately 2.5 ms (i.e., every feedback cycle in the series can have the same duration).

As described above, the controller 240 can use the impedance value as a basis for deciding whether to adjust the power of the output power, and determining an amount to adjust the power of the output power. As described in further detail below, the controller 240 can also be configured to cause, based on the impedance value, the power converter 236 to stop the output power.

As described above, the controller 240 can control operation of the electrosurgical generator 110. Within examples, the controller 240 can be implemented using hardware, software, and/or firmware. For instance, the controller 240 can include one or more processors and a non-transitory computer readable medium (e.g., volatile and/or non-volatile memory) that stores machine language instructions or other executable instructions. The instructions, when executed by the one or more processors, cause the electrosurgical generator 110 to carry out the various operations described herein. The controller 240, thus, can receive data and store the data in the memory as well.

Referring now to FIG. 5, a simplified block diagram of the electrosurgical generator 110 is shown according to another example. In particular, FIG. 5 shows additional components for one example implementation of the block diagram shown in FIG. 2. For instance, as shown in FIG. 5, the power source 234 can include a power supply 534A and a plurality of voltage regulators 534B-534D, and the power converter 236 can include a switched-mode power supply (SMPS) 536A, a modulating gate 536B, a cut-off gate 536C, an output stage 536D, and a cut/coag circuit 536E. Also, as shown in FIG. 5, the controller 240 can include a supervisor microprocessor 540A and a driver microprocessor 540B, and the sensors 238 can include a bipolar sense module 538A, a mono sense module 538B, and a neutral sense module 538C.

In this example, the power supply 534A can receive a grid power 574 (e.g., from a wall outlet), and use the grid power 574 to supply a plurality of powers to various components of the electrosurgical generator 110. For instance, the power supply 534A can include a first output that provides the supply power to the power converter 236 and a second output that provides another power signal to the voltage regulators 534B-534D. Thus, in this example, the power source can be operable to perform an initial power conversion to convert the grid power 574 to the supply power, which the power converter 236 can convert to the output power as described above. The voltage regulators 534B-534D can help regulate the other power signal at a plurality of different voltage levels for operating various components of the electrosurgical generator 110 (e.g., a 12 volt signal, a 5 volt signal, and a 3.3 volt signal).

As shown in FIG. 5, the SMPS 536A of the power converter 236 can receive the supply power from the power source 234 (e.g., via the power supply 534A and the voltage regulator 534C). In an example, the SMPS 536A can provide to the output stage 536D a power signal 578 with a voltage between approximately 5 volts and approximately 83 volts based on a control signal 576 provided by the driver microprocessor 540B to the SMPS 536A. In one implementation, the control signal 576 can be a digital-to-analog (DAC) signal having a voltage between approximately 0 volts and approximately 3 volts for controlling the SMPS 536A. In this way, the controller 240 can set and/or adjust a voltage of the output power provided by the output stage 536D of the power converter 236.

The driver microprocessor 540B can also transmit a modulation signal 580 to the output stage 536D via the modulation gate 536B and the cut-off gate 536C. The modulation gate 536B can use the modulation signal 580 to modulate the output power provided by the output stage 536D (e.g., a MOSFET-based component and/or one or more transformers of the output stage 536D). In this example, the modulation signal 580 causes the output stage 536D to provide the output power with a frequency of approximately 470 kHz. However, as described, the modulation signal 580 can cause the output stage 536D to provide the output power with a different frequency in other examples.

As described above, the controller 240 can set and/or adjust the electrical parameter(s) of the output power based on the mode of operation and/or the one or more inputs received via the user interface 118. As shown in FIG. 5, the user interface 118 can be in communication with the supervisor microprocessor 540A (e.g., via one or more communication terminals 582), and the supervisor microprocessor 540A can be in communication with the driver microprocessor 540B. In this arrangement, the supervisor microprocessor 540A can receive the input(s) from the user interface 118 (e.g., relating to a level of power and/or a mode of operation), the supervisor microprocessor 540A can communicate to the driver microprocessor 540B control data based on the input(s), and the driver microprocessor 540B can transmit the control signal 576 and/or the modulation signal 580 based, at least in part, on the control data.

Within examples, the supervisor microprocessor 540A can perform additional operations. For instance, the supervisor microprocessor 540A can monitor one or more subsystems of the electrosurgical generator 110 to determine when a fault condition occurs. As examples, the supervisor microprocessor 540A is in communication with a temperature sensor 584A, a DAC sensor 584B, and one or more power source sensors 584C. In this arrangement, the supervisor microprocessor 540A can receive sensor information from the temperature sensor 584A, the DAC sensor 584B, and the one or more power source sensors 584C, and determined, based on the sensor information, when the fault condition occurs. As examples, the fault condition can include an overheating condition, a DAC signal fault, and/or a fault relating to the power source 234. Responsive to the supervisor microprocessor 540A determining that a fault condition has occurred, the supervisor microprocessor 540A can cause the cut-off gate 536C of the power converter 236 to stop the output stage 536D providing the output power (e.g., based on the impedance value determined by the controller 240).

In FIG. 5, the supervisor microprocessor 540A can additionally or alternatively control one or more subsystems of the electrosurgical generator 110. For example, in FIG. 5, the supervisor microprocessor 540A is in communication with and operable to control a fan 586 of the electrosurgical generator 110. In one implementation, the supervisor microprocessor 540A can start, stop, speed up, and/or slow down the fan 586 based on sensor information received from the temperature sensor 584A.

In FIG. 5, the electrosurgical generator 110 is operable in a manual bipolar mode, an automatic bipolar mode, and a monopolar mode. As such, the at least one tool input 120 includes a bipolar input 520A, a monopolar input 520B, and a neutral input 520C that can be coupled to the output stage 536D. The bipolar input 520A can couple to the bipolar electrosurgical tool 116 (shown in FIGS. 1-2), the monopolar input 520B can couple to the monopolar electrosurgical tool 112 (shown in FIGS. 1-2), and the neutral input 520C can couple to the dispersive electrode pad 114 (shown in FIGS. 1-2).

As shown in FIG. 5, the bipolar input 520A can include a first terminal and a second terminal that can couple the bipolar electrosurgical tool 116 to the output stage 536D via the bipolar sense module 538A. The bipolar sense module 538A can include one or more current sensors and/or one or more voltage sensors (e.g., the current sensor 244 and the voltage sensor 246 in FIG. 2) for sensing the current and the voltage of the output power, respectively, when operating the electrosurgical generator 110 in a bipolar mode (as described above).

Additionally, the bipolar input 520A can include a third terminal that can be in communication with the controller 240 (e.g., the driver microprocessor 540B) via a bipolar switch sense module 588. The third terminal can also be in communication with the at least one user input device 132 (shown in FIG. 1) on the bipolar electrosurgical tool 116. Thus, the third terminal of the bipolar input 520A and the bipolar switch sense module 588 can be operable to determine and communicate to the controller 240 when the at least one user input device 132 is actuated so that the electrosurgical generator 110 can provide the output power with the corresponding waveform and/or power level for performing electrosurgery in the bipolar mode and using the bipolar electrosurgical tool 116. Within examples, the electrosurgical generator 110 can be additionally or alternatively operated in the bipolar mode using a footswitch 590, which is in communication with the controller 240 (e.g., the driver microprocessor 540B).

When the electrosurgical generator 110 is operated in the manual bipolar mode, an electrosurgical procedure is initiated responsive to the user actuating the at least one user input device 132 and/or the footswitch 590. In particular, when the at least one user input device 132 and/or the footswitch 590 is actuated, the driver microprocessor 540B of the controller 240 can cause the power converter 236 to provide the output power to the bipolar electrosurgical tool 116 via the bipolar sense module 538A and the bipolar input 520A. While the power converter 236 provides the output power, the bipolar sense module 538A senses the current and the voltage of the output power and generates the logarithmic and analog representations of the current and the voltage, as described above. Also, while the power converter 236 provides the output power, the driver microprocessor 540B receives the logarithmic and analog representations of the current and the voltage, and performs the series of feedback cycles to control the power of the output power as described above. In the manual bipolar mode, the power converter 236 can stop providing the output power responsive to the operator ceasing actuation of the at least one user input device 132 and/or the footswitch 590.

As noted above, the electrosurgical generator 110 can also be operated in an automatic bipolar mode. In the automatic bipolar mode, the electrosurgical generator 110 can automatically start and/or stop the electrosurgical procedure (i.e., automatically start and/or stop providing the output power to the bipolar electrosurgical tool 116).

Within examples, the controller 240 can be operable to cause the power converter 236 to automatically start providing the output power at the output stage 536D based on an analysis of a secondary power signal. For instance, as shown in FIG. 5, the bipolar sense module 538A can also be coupled to a secondary power source 594. The secondary power source 594 can provide the secondary power signal to the at least one tool input 120 (e.g., the first terminal and the second terminal of the bipolar input 520A) to apply the secondary power signal to the target tissue 242 (shown in FIG. 2). In general, the secondary power signal has a power and/or a frequency that is less than a power and/or a frequency of the output power. More particularly, the secondary power source can provide the secondary power signal with a power and/or a frequency that is not suitable for performing electrosurgery on the target tissue 242. As an example, the frequency of the secondary power signal can be between approximately 50 kHz and approximately 75 kHz.

The bipolar sense module 538A can sense and communicate to the controller 240 a secondary current of the secondary power signal and sense a secondary voltage of the secondary power signal. The driver microprocessor 540B of the controller 240 can determine a secondary impedance value based on the secondary current and the secondary voltage (e.g., based on Ohm's law). The driver microprocessor 540B of the controller 240 can then cause, based on the secondary impedance value, the power converter 236 to start providing the output power to the bipolar electrosurgical tool 116.

In one example, the driver microprocessor 540B of the controller 240 can perform a comparison of the secondary impedance value to a reference impedance value. If the secondary impedance value is less than the reference impedance value, the driver microprocessor 540B can decide to not start providing the output power. Whereas, if the secondary impedance value is greater than the reference impedance value, the driver microprocessor 540B can decide to start providing the output power. Responsive to the driver microprocessor 540B deciding to start providing the output power, the driver microprocessor 540B can provide the control signal 576 and/or the modulation signal 580 to the power converter 236 to cause the power converter 236 to start providing the output power at the output stage 536D.

Within examples, the driver microprocessor 540B of the controller 240 can automatically stop providing the output power based on an impedance determined based on the output power (i.e., as opposed to the secondary power signal). In one example, the driver microprocessor 540B can determine the impedance value based on the average current value of the output power and the average voltage value of the output power, as described above. The driver microprocessor 540B of the controller 240 can then perform a comparison of the impedance value to a second reference impedance value. If the impedance value is less than the second reference impedance value, the driver microprocessor 540B can decide to continue providing the output power. Whereas, if the impedance value is greater than the second reference impedance value, the driver microprocessor 540B can decide to stop providing the output power. Responsive to the driver microprocessor 540B deciding to stop providing the output power, the driver microprocessor 540B can provide the control signal 576 and/or the modulation signal 580 to the power converter 236 to cause the power converter 236 to stop providing the output power at the output stage 536D.

In another example, the bipolar sense module 538A can additionally or alternatively sense a phase of the impedance of the output power and/or a magnitude of the impedance of the output power. The bipolar sense module 538A can communicate the phase of the impedance and/or the magnitude of the impedance of the output power to the driver microprocessor 540B, and the driver microprocessor 540B can use the phase of the impedance and/or the magnitude of the impedance as a basis for determining when to stop providing the output power. For instance, the driver microprocessor 540B can determine a real component of the impedance based on the phase of the impedance and/or the magnitude of the impedance, and then perform a comparison of the real component of the impedance to the second reference impedance value as described above. This can beneficially help to take into account an impedance and/or a capacitance of the electrical conductor 122 between the bipolar input 520A and the bipolar electrosurgical tool 116.

As also noted above, the electrosurgical generator 110 can be operated in a manual mode. For instance, as shown in FIG. 5, the monopolar input 520B can include a first terminal that can couple the monopolar electrosurgical tool 112 to the output stage 536D via the mono sense module 538B. The mono sense module 538B can include one or more current sensors and/or one or more voltage sensors (e.g., the current sensor 244 and the voltage sensor 246 in FIG. 2) for sensing the current and the voltage of the output power, respectively, when operating the electrosurgical generator 110 in a monopolar mode (as described above).

The monopolar input 520B can also include a second terminal and a third terminal that can be in communication with the controller 240 (e.g., the driver microprocessor 540B) via a cut/coag sense module 592. The second terminal and the third terminal of the monopolar input 520B can also be in communication with respective input devices of the at least one user input device 128 (shown in FIG. 1) on the monopolar electrosurgical tool 112. Thus, the cut/coag sense module 592 can be operable to determine and communicate to the controller 240 when the at least one user input device 128 is actuated so that the electrosurgical generator 110 can provide the output power with the corresponding waveform and/or power level for performing electrosurgery in the cut mode or the coag mode and using the monopolar electrosurgical tool 112.

Responsive to actuating the at least one user input device 128, the driver microprocessor 540B of the controller 240 can also actuate the cut/coag circuit 536E to configure the power converter 236 for the selected mode of operation. For instance, the cut/coag circuit 536E can include one or more electrical components that can facilitate the output stage 536D providing the output power with the one or more electrical parameters that are suitable for operating the electrosurgical generator 110 in a cut mode of operation and/or a coagulation mode of operation. In one example, the driver microprocessor 540B can communicate a second control signal to cause the cut/coag circuit 536E to open and/or close a switch to connect or disconnect the electrical components of the cut/coag circuit 536E.

The neutral input 520C can include a first terminal and a second terminal that can couple to the dispersive electrode pad 114 to the output stage 536D. As such, the neutral input 520C can facilitate returning the electrosurgical energy delivered to the target tissue 242 (shown in FIG. 2) via the monopolar electrosurgical tool 112.

When the electrosurgical generator 110 is operated in the monopolar mode, an electrosurgical procedure is initiated responsive to the user actuating the at least one user input device 128. In particular, when the at least one user input device is actuated, the driver microprocessor 540B of the controller 240 can cause the power converter 236 to provide the output power to the monopolar electrosurgical tool 112 via the monopolar input 520B and return the electrosurgical energy via the neutral input 520C. While the power converter 236 provides the output power, the mono sense module 538B senses the current and the voltage of the output power and generates the logarithmic and analog representations of the current and the voltage, as described above. Also, while the power converter 236 provides the output power, the driver microprocessor 540B receives the logarithmic and analog representations of the current and the voltage, and performs the series of feedback cycles to control the power of the output power as described above. In the monopolar mode, the power converter 236 can stop providing the output power responsive to the operator ceasing actuation of the at least one user input device 128.

Within examples, the electrosurgical generator 110 can be configured to use the secondary power signal from the secondary power source to determine that the dispersive electrode pad 114 sufficiently contacts the patient. For instance, as shown in FIG. 5, the first terminal and the second terminal of the neutral input 520C can be coupled to the neutral sense module 538C. The neutral sense module 538C can include one or more current sensors and/or one or more voltage sensors (e.g., the current sensor 244 and the voltage sensor 246 in FIG. 2) for sensing the secondary current and the secondary voltage of the secondary power signal, respectively, before, during, and/or after providing the output power to the target tissue 242 (as described above).

The neutral sense module 538C can communicate the secondary current and/or the secondary voltage of the second power signal to the controller 240 (e.g., the driver microprocessor 540B), and the controller 240 can determine the secondary impedance value based on the secondary current and the secondary voltage, as described above. The controller 240 can perform a comparison of the secondary impedance value to a third reference impedance value. If the secondary impedance value is greater than the third reference impedance value, the driver microprocessor 540B can decide that a fault condition has not occurred. Whereas, if the secondary impedance value is less than the third reference impedance value, the driver microprocessor 540B can decide that the fault condition has occurred.

Responsive to the driver microprocessor 540B determining that the fault condition has occurred, the driver microprocessor 540B can provide the control signal 576 and/or the modulation signal 580 to the power converter 236 to cause the power converter 236 to stop providing the output power at the output stage 536D. Additionally, the controller 240 (e.g., the driver microprocessor 540B and/or the supervisor microprocessor 540A) can cause an output device 597 to generate at least one of a visual alarm or an audio alarm. In this way, the output device 597 can be configured to generate the at least one of a visual alarm or an audio alarm based on the secondary impedance value.

As shown in FIG. 5, the electrosurgical generator 110 can include a relay control 596 and a plurality of switches 598 that can facilitate operating the electrosurgical generator 110 in the manual bipolar mode, the automatic bipolar mode, and the monopolar mode. For instance, the bipolar input 520A, the monopolar input 520B, and/or the neutral input 520C can each be coupled to the power converter 236 by respective ones of the switches 598. Each switch 598 can be actuated between a closed state in which the switch 598 couples the power converter 236 to the at least one tool input 120 corresponding to the switch 598, and an open state in which the switch 598 decouples the power converter 236 from the at least one tool input 120 corresponding to the switch 598.

The relay control 596 is in communication with the driver microprocessor 540B and the switches 598. In this arrangement, when the at least one user input device 128 is actuated, the driver microprocessor 540B can cause the relay control 596 to actuate the switches 598 such that the monopolar input 520B and the neutral input 520C are coupled to the power converter 236 and the bipolar input 520A is decoupled from the power converter 236. Whereas, when the at least one user input device 132 and/or the footswitch 590 is actuated, the driver microprocessor 540B can cause the relay control 596 to actuate the switches 598 such that the monopolar input 520B and the neutral input 520C are decoupled to the power converter 236 and the bipolar input 520A is coupled from the power converter 236.

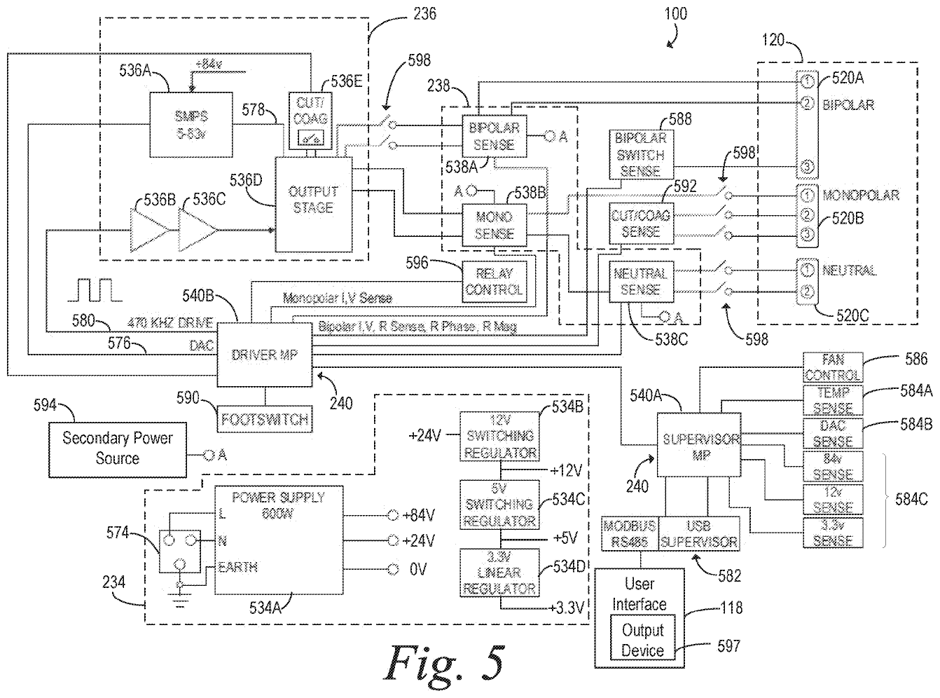

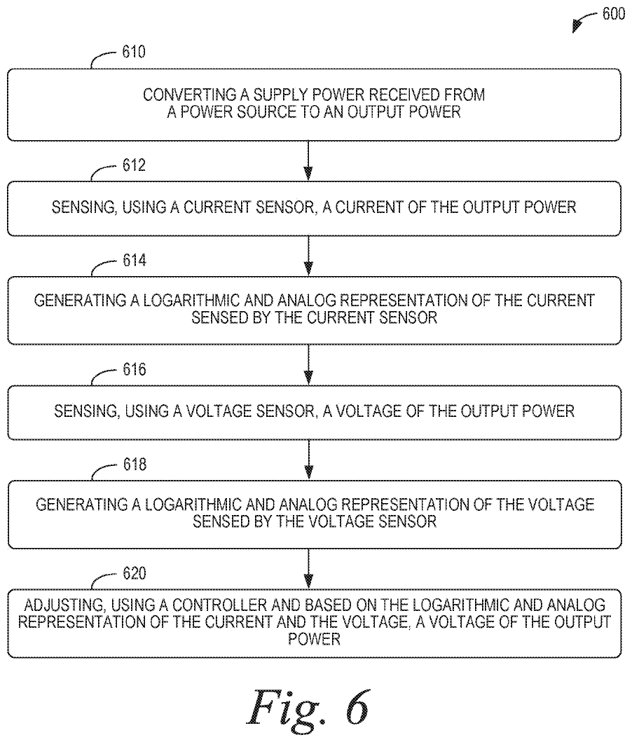

Referring now to FIG. 6, a flowchart for a process 600 of generating electrosurgical energy is shown according to an example. As shown in FIG. 6, the process 600 includes converting a supply power received from a power source to an output power at block 610. The output power is suitable for delivering electrosurgical energy. At block 612, the process 600 includes sensing, using a current sensor, a current of the output power. At block 614, the process 600 includes generating a logarithmic and analog representation of the current sensed by the current sensor. At block 616, the process 600 includes sensing, using a voltage sensor, a voltage of the output power. At block 618, the process 600 includes generating a logarithmic and analog representation of the voltage sensed by the voltage sensor. At block 620, the process 600 includes adjusting, using a controller and based on the logarithmic and analog representation of the current and the voltage, a voltage of the output power.

FIGS. 7-15 depict additional aspects of the process 600 according to further examples. As shown in FIG. 7, sensing the current of the output power at block 612 can include sensing, using a logarithmic RMS current detector, the current of the output power as a RMS current value at block 622. Also, as shown in FIG. 7, sensing the voltage of the output power at block 616 can include sensing, using a logarithmic RMS voltage detector, the voltage of the output power as a RMS voltage value at block 624.

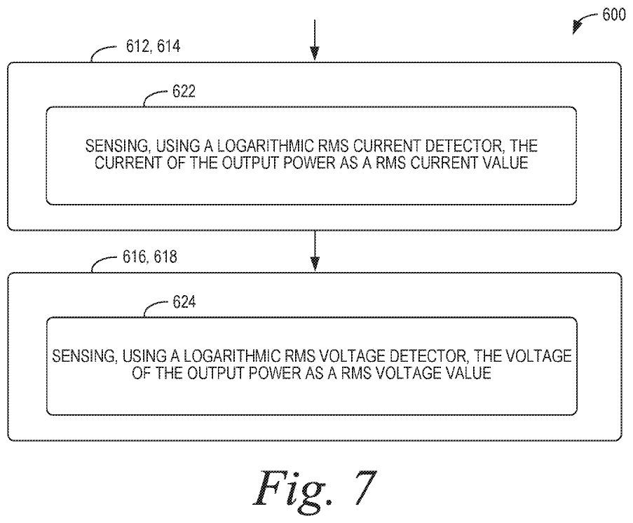

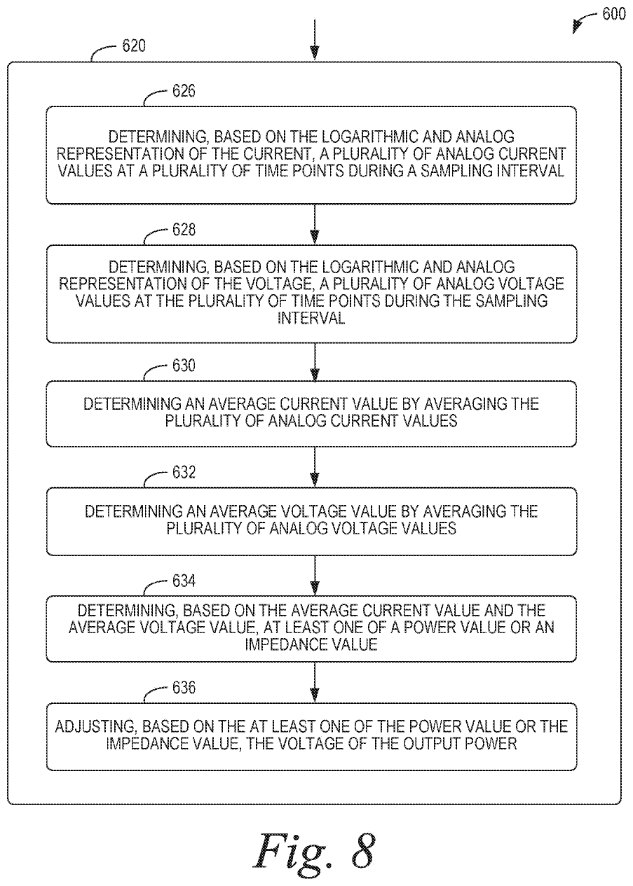

As shown in FIG. 8, adjusting the voltage of the output power at block 620 can include (i) determining, based on the logarithmic and analog representation of the current, a plurality of analog current values at a plurality of time points during a sampling interval at block 626, (ii) determining, based on the logarithmic and analog representation of the voltage, a plurality of analog voltage values at the plurality of time points during the sampling interval at block 628, (iii) determining an average current value by averaging the plurality of analog current values at block 630, (iv) determining an average voltage value by averaging the plurality of analog voltage values at block 632, (v) determining, based on the average current value and the average voltage value, at least one of a power value or an impedance value at block 634, and (vi) adjusting, based on the at least one of the power value or the impedance value, the voltage of the output power at block 636.

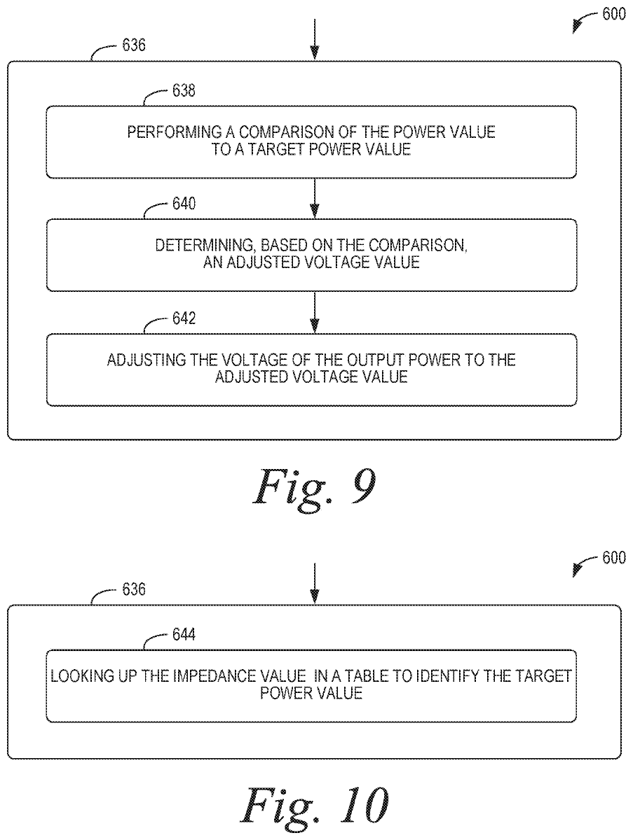

As shown in FIG. 9, adjusting the voltage of the output power at block 636 can further include performing a comparison of the power value to a target power value at block 638, determining, based on the comparison, an adjusted voltage value at block 640, and adjusting the voltage of the output power to the adjusted voltage value at block 642.

As shown in FIG. 10, adjusting the voltage of the output power at block 636 can include looking up the impedance value in a table to identify the target power value that corresponds to the impedance value at block 644.

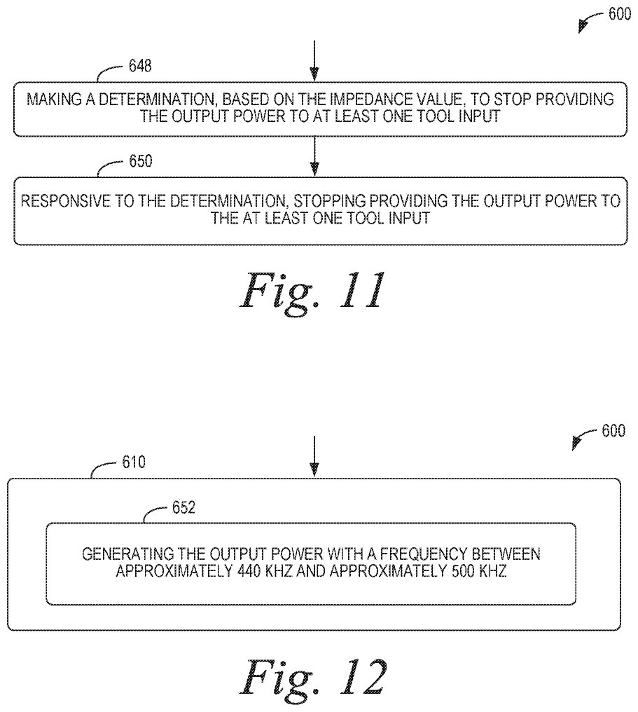

As shown in FIG. 11, the process 600 can further include making a determination, based on the impedance value, to stop providing the output power to at least one tool input at block 648 and, responsive to the determination at block 648, stopping providing the output power to the at least one tool input at block 650.

As shown in FIG. 12, converting the supply power to the output power at block 610 can include generating the output power with a frequency between approximately 440 kHz and approximately 500 kHz at block 652.

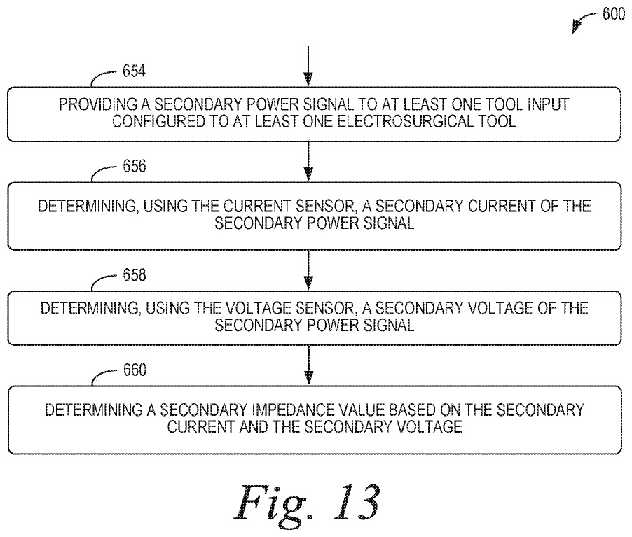

As shown in FIG. 13, the process 600 can further include providing a secondary power signal to at least one tool input configured to at least one electrosurgical tool at block 654. The secondary power signal can have a frequency that is less than a frequency of the output power. The process 600 can also include determining, using the current sensor, a secondary current of the secondary power signal at block 656. Additionally, the process 600 can include determining, using the voltage sensor, a secondary voltage of the secondary power signal at block 658. The process 600 can also include determining a secondary impedance value based on the secondary current and the secondary voltage at block 660.

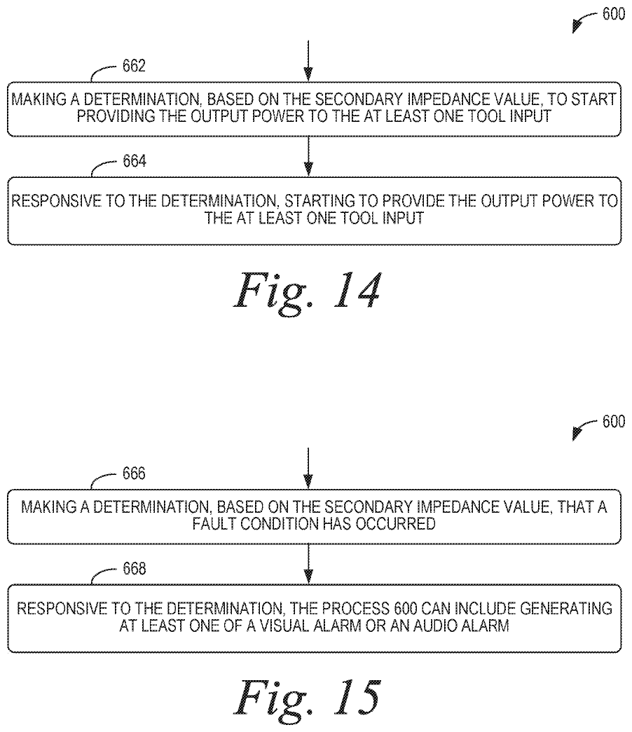

As shown in FIG. 14, the process 600 can further include making a determination, based on the secondary impedance value, to start providing the output power to the at least one tool input at block 662. Responsive to the determination at block 662, the process 600 can include starting to provide the output power to the at least one tool input at block 664.

As shown in FIG. 15, the process 600 can further include making a determination, based on the secondary impedance value, that a fault condition has occurred at block 666. Responsive to the determination at block 666, the process 600 can include generating at least one of a visual alarm or an audio alarm at block 668.

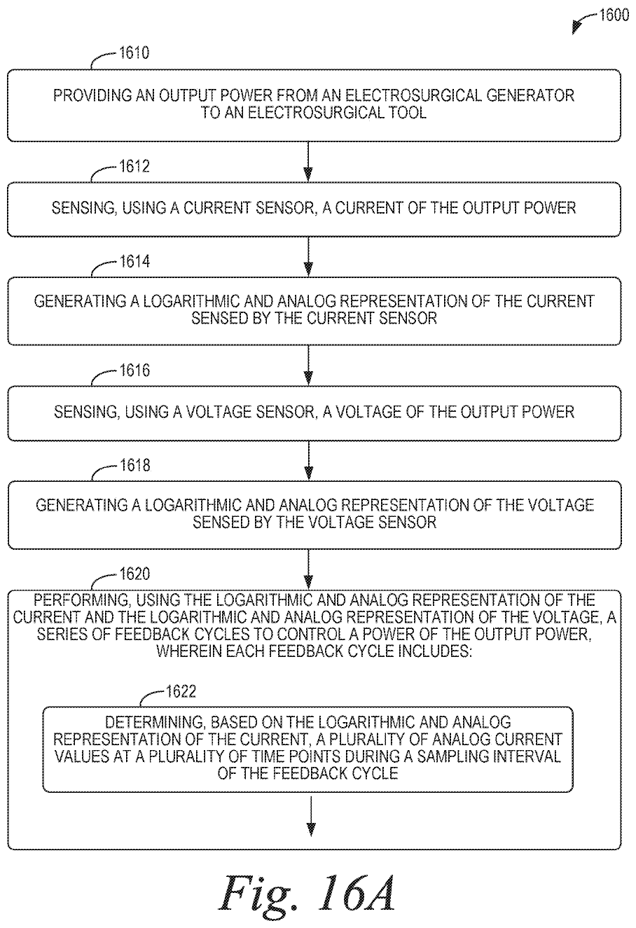

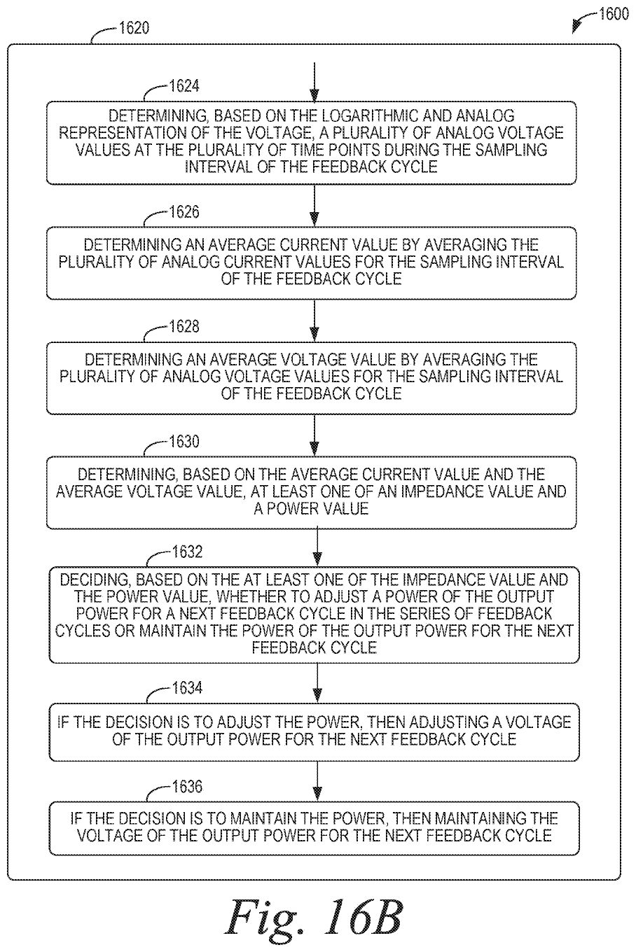

Referring now to FIGS. 16A-16B, a flowchart for a process 1600 of generating electrosurgical energy is shown according to an example. As shown in FIGS. 16A-16B, the process 1600 can include providing an output power from an electrosurgical generator to an electrosurgical tool at block 1610. The process 1600 can also include sensing, using a current sensor, a current of the output power at block 1612 and generating a logarithmic and analog representation of the current sensed by the current sensor at block 1614. The process 1600 can further include sensing, using a voltage sensor, a voltage of the output power at block 1616 and generating a logarithmic and analog representation of the voltage sensed by the voltage sensor at block 1618.

Additionally, the process 1600 can include performing, using the logarithmic and analog representation of the current and the logarithmic and analog representation of the voltage, a series of feedback cycles to control a power of the output power at block 1620. Each feedback cycle can include (i) determining, based on the logarithmic and analog representation of the current, a plurality of analog current values at a plurality of time points during a sampling interval of the feedback cycle at block 1622, (ii) determining, based on the logarithmic and analog representation of the voltage, a plurality of analog voltage values at the plurality of time points during the sampling interval of the feedback cycle at block 1624, (iii) determining an average current value by averaging the plurality of analog current values for the sampling interval of the feedback cycle at block 1626, (iv) determining an average voltage value by averaging the plurality of analog voltage values for the sampling interval of the feedback cycle at block 1628, (v) determining, based on the average current value and the average voltage value, at least one of an impedance value and a power value at block 1630, (vi) deciding, based on the at least one of the impedance value and the power value, whether to adjust a power of the output power for a next feedback cycle in the series of feedback cycles or maintain the power of the output power for the next feedback cycle at block 1632, (vii) if the decision is to adjust the power at block 1632, then adjusting a voltage of the output power for the next feedback cycle at block 1634, and (viii) if the decision is to maintain the power at block 1632, then maintaining the voltage of the output power for the next feedback cycle at block 1636. For at least one feedback cycle in the series of feedback cycles, the decision at block 1632 is to adjust the power of the output power.

One or more of the blocks shown in FIGS. 6-16B may represent a module, a segment, or a portion of program code, which includes one or more instructions executable by a processor for implementing specific logical functions or steps in the process. The program code may be stored on any type of computer readable medium or data storage, for example, such as a storage device including a disk or hard drive. Further, the program code can be encoded on a computer-readable storage media in a machine-readable format, or on other non-transitory media or articles of manufacture. The computer readable medium may include non-transitory computer readable medium or memory, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a tangible computer readable storage medium, for example.

In some instances, components of the devices and/or systems described herein may be configured to perform the functions such that the components are actually configured and structured (with hardware and/or software) to enable such performance. Example configurations then include one or more processors executing instructions to cause the system to perform the functions. Similarly, components of the devices and/or systems may be configured so as to be arranged or adapted to, capable of, or suited for performing the functions, such as when operated in a specific manner.

Further, the disclosure comprises examples according to the following clauses:

Clause 1: In an example, an electrosurgical generator is described. The electrosurgical generator includes a power converter configured to convert a supply power received from a power source to an output power. The output power is suitable for delivering electrosurgical energy. The electrosurgical generator also includes a current sensor configured to sense a current of the output power and generate a logarithmic and analog representation of the current, and a voltage sensor configured to sense a voltage of the output power and generate a logarithmic and analog representation of the voltage. The electrosurgical generator further includes a controller configured to: (i) receive the logarithmic and analog representation of the current sensed by the current sensor, (ii) receive the logarithmic and analog representation of the voltage sensed by the voltage sensor, and (iii) adjust, based on the logarithmic and analog representation of the current and the voltage, a voltage of the output power.

Clause 2: In another example, a method of generating electrosurgical energy is described. The method includes converting a supply power received from a power source to an output power, wherein the output power is suitable for delivering electrosurgical energy. The method also includes sensing, using a current sensor, a current of the output power. The method further includes generating a logarithmic and analog representation of the current sensed by the current sensor. The method also includes sensing, using a voltage sensor, a voltage of the output power. Additionally, the method includes generating a logarithmic and analog representation of the voltage sensed by the voltage sensor. The method also includes adjusting, using a controller and based on the logarithmic and analog representation of the current and the voltage, a voltage of the output power.

Clause 3: In another example, a method of generating electrosurgical energy is described. The method includes providing an output power from an electrosurgical generator to an electrosurgical tool. The method also includes sensing, using a current sensor, a current of the output power, and generating a logarithmic and analog representation of the current sensed by the current sensor. The method further includes sensing a voltage of the output power and generating a logarithmic and analog representation of the voltage. The method further includes performing, using the logarithmic and analog representation of the current and the logarithmic and analog representation of the voltage, a series of feedback cycles to control a power of the output power. Each feedback cycle includes: (i) determining, based on the logarithmic and analog representation of the current, a plurality of analog current values at a plurality of time points during a sampling interval of the feedback cycle, (ii) determining, based on the logarithmic and analog representation of the voltage, a plurality of analog voltage values at the plurality of time points during the sampling interval of the feedback cycle, (iii) determining an average current value by averaging the plurality of analog current values for the sampling interval of the feedback cycle, (iv) determining an average voltage value by averaging the plurality of analog voltage values for the sampling interval of the feedback cycle, (v) determining, based on the average current value and the average voltage value, at least one of an impedance value and a power value, (vi) deciding, based on the at least one of the impedance value and the power value, whether to adjust a power of the output power for a next feedback cycle in the series of feedback cycles or maintain the power of the output power for the next feedback cycle, (vii) if the decision is to adjust the power, then adjusting a voltage of the output power for the next feedback cycle, and (viii) if the decision is to maintain the power, then maintaining the voltage of the output power for the next feedback cycle. For at least one feedback cycle in the series of feedback cycles, the decision is to adjust the power of the output power.

The description of the different advantageous arrangements has been presented for purposes of illustration and description, and is not intended to be exhaustive or limited to the examples in the form disclosed. Many modifications and variations will be apparent to those of ordinary skill in the art. Further, different advantageous examples may describe different advantages as compared to other advantageous examples. The example or examples selected are chosen and described in order to explain the principles of the examples, the practical application, and to enable others of ordinary skill in the art to understand the disclosure for various examples with various modifications as are suited to the particular use contemplated.

What is claimed is:

1. An electrosurgical generator, comprising:
a power converter configured to convert a supply power received from a power source to an output power, wherein the output power is suitable for delivering electrosurgical energy;
a current sensor configured to sense a current of the output power and generate a logarithmic and analog representation of the current;
a voltage sensor configured to sense a voltage of the output power and generate a logarithmic and analog representation of the voltage; and
a controller configured to:
receive the logarithmic and analog representation of the current sensed by the current sensor,
receive the logarithmic and analog representation of the voltage sensed by the voltage sensor, and
adjust, based on the logarithmic and analog representation of the current and the voltage, a voltage of the output power.

2. The electrosurgical generator of claim 1, wherein the current sensor is a logarithmic root-mean-square (RMS) current detector configured to sense the current of the output power as a RMS current value, and
wherein the voltage sensor is a logarithmic RMS voltage detector configured to sense the voltage of the output power as a RMS voltage value.

3. The electrosurgical generator of claim 1, wherein, to adjust the voltage of the output power, the controller is configured to:
determine, based on the logarithmic and analog representation of the current, a plurality of analog current values at a plurality of time points during a sampling interval,
determine, based on the logarithmic and analog representation of the voltage, a plurality of analog voltage values at the plurality of time points during the sampling interval,
determine an average current value by averaging the plurality of analog current values,
determine an average voltage value by averaging the plurality of analog voltage values,
determine, based on the average current value and the average voltage value, at least one of a power value or an impedance value, and
adjust, based on the at least one of the power value or the impedance value, the voltage of the output power.

4. The electrosurgical generator of claim 3, wherein, to adjust the voltage of the output power, the controller is further configured to:
perform a comparison of the power value to a target power value,
determine, based on the comparison, an adjusted power value, and
adjust the voltage of the output power to the adjusted power value.

5. The electrosurgical generator of claim 3, wherein to adjust the voltage of the output power, the controller is configured to:
look up the impedance value and the power value in a table to identify an adjusted voltage value that corresponds to the impedance value and the power value, and
adjust the voltage of the output power to the adjusted voltage value.

6. The electrosurgical generator of claim 3, wherein the sampling interval is a portion of a time window,
wherein the time window is between approximately 2 milliseconds (ms) to approximately 2.5 ms, and
wherein the sampling interval is approximately 132 microseconds.

7. The electrosurgical generator of claim 3, wherein the controller is further configured to cause, based on the impedance value, the power converter to stop the output power.

8. The electrosurgical generator of claim 1, wherein the output power has a frequency between approximately 440 kilohertz (kHz) and approximately 500 kHz.

9. The electrosurgical generator of claim 1, further comprising at least one tool input configured to couple the power converter to at least one electrosurgical tool,
wherein the power converter is configured to provide a secondary power signal to at least one tool input,
wherein the secondary power signal has a frequency that is less than a frequency of the output power,
wherein the current sensor is configured to determine a secondary current of the secondary power signal,
wherein the voltage sensor is configured to determine a secondary voltage of the secondary power signal, and
wherein the controller is configured to determine a secondary impedance value based on the secondary current and the secondary voltage.

10. The electrosurgical generator of claim 9, wherein the controller is configured to cause, based on the secondary impedance value, the power converter to start providing the output power to the at least one tool input.

11. The electrosurgical generator of claim 9, further comprising an output device configured to generate at least one of a visual alarm or an audio alarm based on the secondary impedance value.

12. The electrosurgical generator of claim 9, wherein the frequency of the secondary power signal is between approximately 50 kHz and approximately 75 kHz.

13. A method of generating electrosurgical energy, comprising:
converting a supply power received from a power source to an output power, wherein the output power is suitable for delivering electrosurgical energy;
sensing, using a current sensor, a current of the output power;
generating a logarithmic and analog representation of the current sensed by the current sensor;
sensing, using a voltage sensor, a voltage of the output power;
generating a logarithmic and analog representation of the voltage sensed by the voltage sensor; and
adjusting, using a controller and based on the logarithmic and analog representation of the current and the voltage, a voltage of the output power.

14. The method of claim 13, wherein sensing the current of the output power and generating the logarithmic and analog representation of the current comprises sensing, using a logarithmic root-mean-square (RMS) current detector, the current of the output power as a RMS current value, and
wherein sensing the voltage of the output power generating the logarithmic and analog representation of the current comprises sensing, using a logarithmic RMS voltage detector, the voltage of the output power as a RMS voltage value.

15. The method of claim 13, wherein adjusting the voltage of the output power comprises:
determining, based on the logarithmic and analog representation of the current, a plurality of analog current values at a plurality of time points during a sampling interval,
determining, based on the logarithmic and analog representation of the voltage, a plurality of analog voltage values at the plurality of time points during the sampling interval,
determining an average current value by averaging the plurality of analog current values,
determining an average voltage value by averaging the plurality of analog voltage values,
determining, based on the average current value and the average voltage value, at least one of a power value or an impedance value, and
adjusting, based on the at least one of the power value or the impedance value, the voltage of the output power.

16. The method of claim 15, wherein adjusting the voltage of the output power comprises:
performing a comparison of the power value to a target power value,
determining, based on the comparison, an adjusted voltage value, and
adjusting the voltage of the output power to the adjusted voltage value.

17. The method of claim 16, wherein adjusting the voltage of the output power further comprises:
looking up the impedance value in a table to identify the target power value.

18. The method of claim 15, wherein the sampling interval is a portion of a time window,
wherein the time window is between approximately 2 milliseconds (ms) to approximately 2.5 ms, and
wherein the sampling interval is approximately 132 microseconds.

19. The method of claim 15, further comprising:
making a determination, based on the impedance value, to stop providing the output power to at least one tool input; and
responsive to the determination, stopping providing the output power to the at least one tool input.

20. The method of claim 13, wherein converting the supply power to the output power comprises generating the output power with a frequency between approximately 440 kilohertz (kHz) and approximately 500 kHz.

21. The method of claim 13, further comprising providing a secondary power signal to at least one tool input configured to at least one electrosurgical tool, wherein the secondary power signal has a frequency that is less than a frequency of the output power,
determining, using the current sensor, a secondary current of the secondary power signal;
determining, using the voltage sensor, a secondary voltage of the secondary power signal; and
determining a secondary impedance value based on the secondary current and the secondary voltage.

22. The method of claim 21, further comprising:
making a determination, based on the secondary impedance value, to start providing the output power to the at least one tool input; and
responsive to the determination, starting to provide the output power to the at least one tool input.

23. The method of claim 21, further comprising:
making a determination, based on the secondary impedance value, that a fault condition has occurred; and
responsive to the determination, generating at least one of a visual alarm or an audio alarm.

24. The method of claim 21, wherein the frequency of the secondary power signal is between approximately 50 kHz and approximately 75 kHz.

25. A method of generating electrosurgical energy, comprising:
providing an output power from an electrosurgical generator to an electrosurgical tool;
sensing, using a current sensor, a current of the output power;
generating a logarithmic and analog representation of the current sensed by the current sensor;
sensing, using a voltage sensor, a voltage of the output power;
generating a logarithmic and analog representation of the voltage sensed by the voltage sensor; and
performing, using the logarithmic and analog representation of the current and the logarithmic and analog representation of the voltage, a series of feedback cycles to control a power of the output power, wherein each feedback cycle comprises:
determining, based on the logarithmic and analog representation of the current, a plurality of analog current values at a plurality of time points during a sampling interval of the feedback cycle,
determining, based on the logarithmic and analog representation of the voltage, a plurality of analog voltage values at the plurality of time points during the sampling interval of the feedback cycle,
determining an average current value by averaging the plurality of analog current values for the sampling interval of the feedback cycle,
determining an average voltage value by averaging the plurality of analog voltage values for the sampling interval of the feedback cycle,
determining, based on the average current value and the average voltage value, at least one of an impedance value and a power value,
deciding, based on the at least one of the impedance value and the power value, whether to adjust a power of the output power for a next feedback cycle in the series of feedback cycles or maintain the power of the output power for the next feedback cycle,
if the decision is to adjust the power, then adjusting a voltage of the output power for the next feedback cycle, and
if the decision is to maintain the power, then maintaining the voltage of the output power for the next feedback cycle,
wherein, for at least one feedback cycle in the series of feedback cycles, the decision is to adjust the power of the output power.
